(19) United States
(12) Patent Application Publication
(10) Pub. No.: US 2024/0294949 A1
(43) Pub. Date: Sep. 5, 2024

(54) METHOD OF TREATMENT OF ANIMAL MANURE FOR BIOGAS PRODUCTION

(71) Applicant: Lystek International Corp., Cambridge (CA)

(72) Inventors: Michael BESWICK, Cambridge (CA); Ajay SINGH, Cambridge (CA); Hanna BENNETT, Cambridge (CA)

(73) Assignee: Lystek International Corp., Cambridge (CA)

(21) Appl. No.: 18/594,264

(22) Filed: Mar. 4, 2024

Related U.S. Application Data
(60) Provisional application No. 63/487,913, filed on Mar. 2, 2023.

(51) Int. Cl. C12P 5/02

(52) U.S. Cl. CPC C12P 5/023

(57) ABSTRACT

A method of processing an animal manure that includes biosolids to provide an output animal manure digestate and an output biogas. The method includes heating the animal manure to a predetermined hydrolysis temperature for a preselected hydrolysis time period. In addition, a pH of the animal manure is caused to be a target hydrolysis pH. The biosolids are subjected to shearing, to produce a hydrolyzed animal manure digestate. After the hydrolysis time period, the hydrolyzed animal manure digestate is subjected to anaerobic digestion at a predetermined anaerobic digestion temperature for a preselected anaerobic digestion time period, to produce the output animal manure digestate and the output biogas.

### CROSS-REFERENCE TO RELATED APPLICATION

This application claims the benefit of U.S. Provisional Patent Application No. 63/487,913, filed on Mar. 2, 2023, the entirety of which provisional patent application is hereby incorporated herein by reference.

### FIELD OF THE INVENTION

The present invention is a method of treatment of animal manure to produce enhanced biogas therefrom.

### BACKGROUND OF THE INVENTION

Large volumes of raw animal manure are produced worldwide, and the volumes are expected to increase. For centuries animal manures have been a traditional source of nutrients in agriculture. However, disposal of animal manure has become an environmental problem recently, due to increased concentration of animal production within small geographic areas. The application of excessive amounts of manure to farmland can lead to accumulation of nutrients in soils with potential for surface and groundwater pollution. Gases emitted (e.g., NH₃, CH₄, N₂O) resulting from the breakdown of animal manure are released during subsequent storage and conventional spreading of manure on land.

In general, animal manures are usually a mixture of feces, urine, discarded bedding, and waste feed, with variable water content. There are significant differences depending on the species of animal. Therefore, some manure treatment technologies can be more suitable than others to handle manure depending on whether the manure is in solid, semi-solid, slurry, or liquid forms.

Anaerobic digestion (AD) is a biological process occurring in low oxygen environments where microorganisms convert organic matter into methane (CH₄), carbon dioxide (CO₂), hydrogen sulfide (H₂S), ammonia (NH₃), volatile organic compounds, and a nutrient-rich sludge. In the prior art, AD is widely used to stabilize organic wastes from industrial and municipal wastewater biosolids by reducing pathogens and odor emissions. Additionally, AD is used to produce energy through biogas/methane generation.

AD of organic material occurs when oxygen is limited, and a proportion of the organic material is biologically converted to biogas. Anaerobic microbes, particularly methanogens capable of converting organic components to methane and other gases, are essential to treating animal manures to form a more stabilized sludge with reduced solids and pathogen load.

Anaerobic digestion is a natural process conventionally utilized for biological treatment of animal manures, typically, in open or covered storage facilities and lagoons. Open anaerobic lagoons are widely used across North America and Europe to store and treat wastewater generated from confined swine and cattle operations. Open anaerobic lagoons reduce the nitrogen content of the material through NH₃ volatilization.

However, many animal manures tend to include substantial amounts of fibrous material (e.g., cellulose and lignin) that is not easily biodegraded via anaerobic digestion. The slow hydrolysis of cellulose and lignin is the rate-controlling step in digestibility of the animal manures. The result is that, in prior art anaerobic digestion processing of animal manures, the fibrous material is only partially biodegraded, at best.

In the prior art, municipal wastewater treatments are known in which the biodegradation of biosolids is promoted using various techniques (e.g., as disclosed in U.S. Pat. No. 11,420,890). In general, however, the waste material treated at a municipal wastewater treatment facility does not include substantial amounts of fibrous material.

In summary, the conventional treatments of animal manures are generally minimal, and tend to be ineffective, achieving only minimal reductions in the pollution from the animal manures.

### SUMMARY OF THE INVENTION

For the foregoing reasons, there is a need for enhanced treatment methods for animal manure.

In its broad aspect, the invention provides a method of processing an animal manure that includes biosolids to provide an output animal manure digestate and an output biogas. The method include storing the animal manure for a first preselected time period at a first predetermined temperature, to at least partially liquefy the biosolids to produce an animal manure digestate, and to generate biogas.

After the first preselected time period, the animal manure digestate is heated to a second predetermined temperature for a second preselected time period. Also, a pH of the animal manure digestate is caused to be a preselected target pH. The animal manure digestate is subjected to shearing, to produce a hydrolyzed animal manure digestate.

After the second preselected time period, the hydrolyzed animal manure digestate is stored for a third preselected time period, at a third predetermined temperature, to produce the output animal manure digestate, and to generate the output biogas.

In another of its aspects, the invention provides a method of processing an animal manure that includes biosolids to provide an output animal manure digestate and an output biogas. The method includes subjecting the animal manure to a first anaerobic digestion at a first predetermined temperature for a first preselected time period, to at least partially liquefy the biosolids to produce an animal manure digestate, and to generate biogas.

After the first preselected time period, the animal manure digestate is heated to a second predetermined temperature for a second preselected time period. Also, a pH of the animal manure digestate is caused to be a target pH. The animal manure digestate is also subjected to shearing, to produce a hydrolyzed animal manure digestate.

After the second preselected time period, the hydrolyzed animal manure digestate is subjected to a second anaerobic digestion at a third predetermined temperature for a third preselected time period, to produce the output animal manure digestate, and to generate the output biogas.

In yet another of its aspects, the invention provides a method of processing an animal manure that includes biosolids to provide an output animal manure digestate and an output biogas. The method includes heating the animal manure to a predetermined hydrolysis temperature for a preselected hydrolysis time period. Also, a pH of the animal manure is caused to be a target hydrolysis pH. The biosolids are subjected to shearing for the preselected hydrolysis time period, to produce a hydrolyzed animal manure digestate.

After the preselected hydrolysis time period, the hydrolyzed animal manure digestate is subjected to anaerobic digestion at a predetermined anaerobic digestion temperature for a preselected anaerobic digestion time period, to produce the output animal manure digestate, and to generate the output biogas.

### BRIEF DESCRIPTION OF THE DRAWINGS

The invention will be better understood with reference to the attached drawings, in which:

FIG. 1 is a schematic illustration of an embodiment of a method of the invention; and

FIG. 2 is a schematic illustration of an alternative embodiment of a method of the invention.

### DETAILED DESCRIPTION

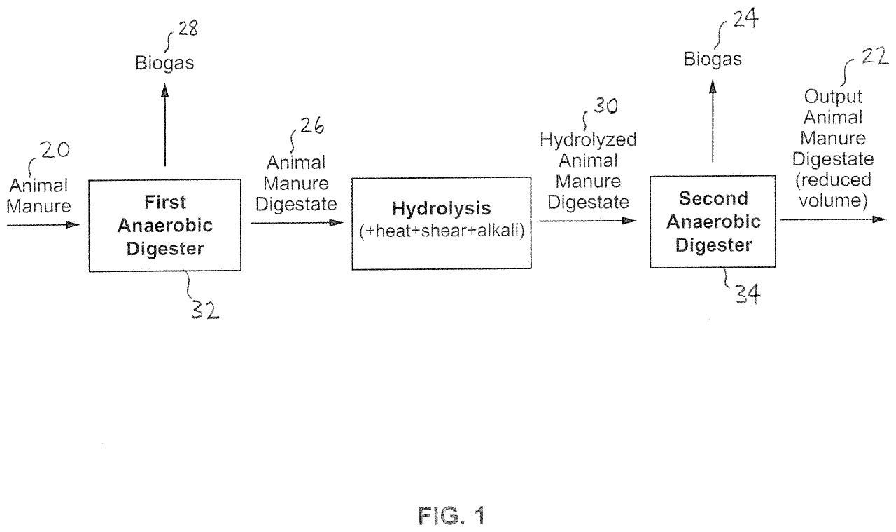

In the attached drawings, like reference characters designate corresponding elements throughout. Reference is first made to FIG. 1 to describe an embodiment of a method in accordance with the invention. As will be described, the method of the invention is a method of processing an animal manure 20 that includes biosolids to provide an output animal manure digestate 22 and an output biogas 24 (FIG. 1).

In one embodiment, the method of the invention preferably includes, first, storing the animal manure for a first preselected time period, at a first predetermined temperature, in order to produce an animal manure digestate 26, and to generate biogas 28.

After the first preselected time period, the animal manure digestate preferably is heated to a second predetermined temperature for a second preselected time period. Preferably, a pH of the animal manure digestate 26 is caused to be a preselected target pH. The animal manure digestate 26 is also subjected to shearing, e.g., by a mixer device, to produce a hydrolyzed animal manure digestate 30.

After the second preselected time period, the hydrolyzed animal manure digestate 30 is stored for a third preselected time period, at a third predetermined temperature, to produce the output animal manure digestate 22, and to generate the output biogas 24.

It will be understood that the various steps of the method of the invention may be carried out in one or more vessels. For example, in practice, it may be preferable to carry out all the steps in a single vessel. For clarity of illustration, in FIG. 1, a “first anaerobic digester” 32 and a “second anaerobic digester” 34 are identified.

In effect, the animal manure preferably is subjected to anaerobic digestion at a first predetermined temperature for a first preselected time period, to liquefy at least part of the biosolids, to provide the animal manure digestate 26, which is then subjected to the thermal-alkaline-mechanical process, i.e., hydrolysis. In one embodiment, the hydrolyzed animal manure digestate 30, after further anaerobic digestion, provides an enhanced biogas, referred to as the output biogas 22. Those skilled in the art would also appreciate that the biogas 28 produced from the first anaerobic digester 32 is not enhanced biogas.

In one embodiment:
the first predetermined temperature preferably is between 35° C. and 40° C.;
the first preselected time period preferably is between two and 40 days;
the second predetermined temperature preferably is between 70° C. and 80° C.;
the preselected target pH preferably is between 8.5 and 9.5;
the second preselected time period preferably is between five minutes and 15 minutes;
the third predetermined temperature preferably is between 35° C. and 40° C.; and
the third preselected time period preferably is between two and 40 days;

Those skilled in the art would appreciate that, in the previously described embodiment, mesophilic anaerobic digesters may be used. However, alternatively, one or both of the anaerobic digesters utilized may be thermophilic. For example, in an alternative embodiment, the first predetermined temperature preferably is between 55° C. and 60° C. Also, in another alternative embodiment, the third predetermined temperature preferably is between 55° C. and 60° C.

The thermal-alkaline-mechanical process of the invention, which produces the hydrolyzed animal manure digestate 30, breaks down the cellular components converting macromolecules present in the organic portion of the biosolids into simpler compounds that are more amenable to biodegradation. Alternatively, only heat and alkali is used as described above to hydrolyze the cellular components into simpler organic compounds.

The output animal manure digestate 22 is a high solids liquid product that is made with the process of the invention can be pumped and transported using conventional liquid handling equipment, and stored as a concentrated liquid material. The biogas 28 and the enhanced biogas 24 may be captured and utilized, as is known in the art.

Cellular components and nutrients in the processed biosolids resulting from the thermal-alkaline-mechanical process or the thermal-alkaline process of the invention are comparatively more available for utilization by anaerobic microorganisms. The thermal-alkaline-mechanical process or the thermal-alkaline process of the invention generates hydrolyzed material 30, with high quantities of soluble carbon, which can be used as an improved anaerobic digester feedstock. The hydrolyzed feedstock 30 has improved biodegradability, in which biologically recalcitrant organic compounds in the material are made more amenable to digestion, and subsequently can enhance overall anaerobic digester kinetics.

It is believed that the process of the invention produces a product 30 that increases the degree and rate of biodegradability in AD through several mechanisms:
Particle size reduction increasing the surface area of organics;
Feedstock homogenization and viscosity reduction;
Solubilization of recalcitrant organic carbon that would otherwise go undigested in conventional anaerobic digesters.

Improved biodegradability in anaerobic digesters provides:
Generation of additional biogas;
Reduction of solids requiring further management;
More rapid generation of biogas;
Shorter hydraulic retention times and smaller digesters;
Reduction of energy requirements for pumping and mixing operations in, out, and within the anaerobic digester.

The process of the invention minimizes the energy inputs required for treatment and transportation of residuals by operating at a higher solid concentration, while maintaining the homogeneous liquid properties of the material. This promotes efficient processing, product handling, storage, and transportation.

Because the process of the invention increases the temperature of the material within the reactor vessel, the residual heat from processing partially offsets heat requirements of the anaerobic digester(s) allowing for additional energy savings. In addition, the operating conditions of the process of the invention tend to have lower greenhouse gas (GHG) emissions when compared to the biosolids treatment practices of the prior art, such as heat drying or incineration, as temperatures are lower and treatment vessels are closed.

Implementing the process of the invention and biogas recovery systems on a livestock farm can significantly reduce or eliminate the usage of fossil fuels, such as propane or diesel, and correspondingly reduce GHG emissions. Those skilled in the art would appreciate that biogas generated can be upgraded into renewable natural gas (RNG) or utilized for combined heat and power applications.

Many livestock farms are in remote rural areas that lack municipal service connections and therefore rely on fossil fuels such as propane and diesel for typical farming operations. Biogas recovered from the livestock manure could be used to power various equipment, e.g., grain dryers, farming equipment, or on-farm vehicles to create a more economically and environmentally sustainable operation.

Thus, processes that can reduce the overall mass and volume of manure while simultaneously generating a product for green energy are useful and enhance agricultural sustainability.

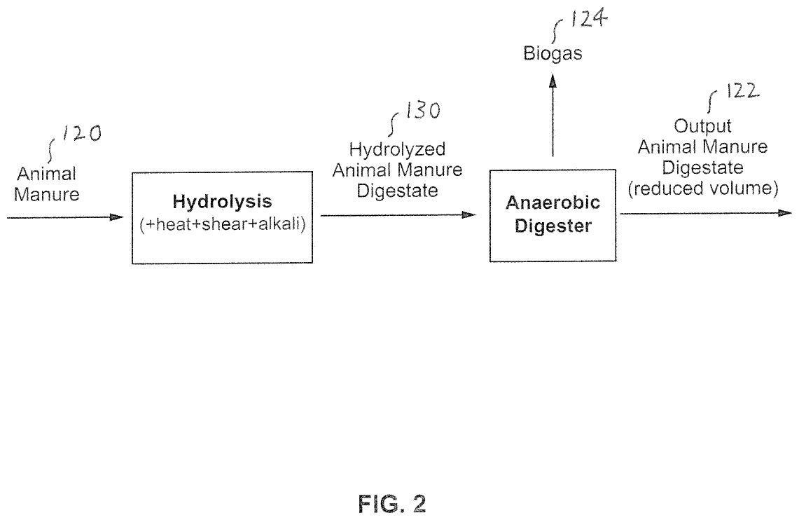

In an alternative embodiment, illustrated in FIG. 2, the invention provides a method of processing an animal manure 120 that includes biosolids to provide an output animal manure digestate 122 and an output biogas 124. The method preferably includes, first, heating the animal manure to a predetermined hydrolysis temperature for a preselected hydrolysis time period, causing a pH of the animal manure 120 to be a target hydrolysis pH, and subjecting the biosolids to shearing for the preselected hydrolysis time period, to produce a hydrolyzed animal manure digestate 130.

After the preselected hydrolysis time period, the hydrolyzed animal manure digestate 130 preferably is subjected to anaerobic digestion at a predetermined anaerobic digestion temperature for a preselected anaerobic digestion time period, to produce the output animal manure digestate 122, and to generate the output biogas 124.

It will be understood that the predetermined hydrolysis temperature and the preselected hydrolysis time period preferably correspond to the second predetermined temperature and the second preselected time period respectively. The second predetermined temperature and the second preselected time period are described above, in connection with the first embodiment of the method of the invention, illustrated in FIG. 1. Accordingly, the predetermined hydrolysis temperature preferably is between 70° C. and 80° C., and the preselected hydrolysis time period preferably is between five minutes and 15 minutes. Also, the target hydrolysis pH of the process schematically illustrated in FIG. 2 corresponds to the target pH of the first embodiment of the method of the invention, and preferably is between 8.5 and 9.5.

It will also be understood that the predetermined anaerobic digestion temperature and the preselected anaerobic digestion time period of the second embodiment preferably correspond to the third predetermined temperature and the third preselected time period respectively, also described above. Accordingly, the predetermined anaerobic digestion temperature preferably is between 35° C. and 40° C., and the preselected anaerobic digestion time period preferably is between two and 40 days. Alternatively, the predetermined anaerobic digestion temperature may be between 55° C. and 60° C.

The method of the invention was used in processing animal manures from different animals, as described below.

### Example I

Beef cattle manure samples were analyzed and treated using the process of the invention. As noted above, the method of the invention includes addition of heat, alkali, and high shear mixing in a vessel to achieve a high-solid liquefied product that can be handled, stored, and further utilized substantially as a liquid using conventional equipment.

The manure samples were stored in a water bath at 38° C. for 5 weeks in anaerobic conditions to remove easily biodegradable compounds. This was done to evaluate the impact of the process of the invention on relatively recalcitrant, hard to degrade organic substrate present in the cow manure samples. Half of the material was then treated with the process of the invention, and the other half was stored as an untreated control for biological methane potential (BMP) tests.

For treatment, manure samples were first heated to 80° C. in a water bath, in a reactor. Thereafter, the heated sample (i.e., heated to 80° C.) was transferred to a heavy duty 1000-watt motor blender (Ninja professional blender 1000, model BL610C), built with variable speed controls. Next, 93 kg of 45% KOH per 1 tonne of dry manure solids was added to the heated sample in the blender jar to bring the pH of the material to 9.5 and then the mixture was sheared at 4,600 rpm for 15 minutes.

Untreated and treated samples of a beef cattle manure were obtained. The “untreated” sample corresponds to the animal manure digestate 26, i.e., prior to being subjected to the thermal-alkaline-mechanical process of the invention. The “treated” sample corresponds to the hydrolyzed animal manure digestate 30.

The “untreated” sample is a sample of the digestate after anaerobic digestion. As noted above, utilizing anaerobic digestion alone is known in the prior art. The “untreated” sample is therefore, in effect, representative of the digestate from anaerobic digestion of animal manure in the prior art. The “treated” sample enables the effect of the thermal-alkaline-mechanical processed the invention to be determined.

The treated sample is more homogeneous than the untreated sample, indicating that the process of the invention efficiently homogenizes and hydrolyzes the manure. This is associated with hydrolysis of long chain components including carbohydrates, proteins, and lipids. Analysis of untreated and treated beef cattle manure samples is shown in Table 1.

TABLE 1
Analysis of untreated and treated beef cattle manure

Analysis

| | Untreated beef cattle | Treated beef cattle |
|---|---|---|
| | manure | manure |
| Total solids (%) | 14.6 | 14.2 |
| Volatile solids (%) | 12.0 | 11.1 |
| Ash (%) | 17.7 | 21.6 |

A biomethane potential (BMP) test using a standard protocol, at a third-party analytical lab, was conducted. Mean gas quality and concentrations was measured over the course of the 28-day experiment. Total solids, volatile solids, and ash content were determined according to DIN EN 12880 and DIN EN 12879 and used as reference parameters for gas production. The loading rate was 3 kg total solids/m³. The testing temperature was 38° C. for the duration of analysis. Microcrystalline cellulose was analyzed as a positive control to ensure biological activity of the inoculum. Methane (CH₄), carbon dioxide (CO₂), and hydrogen sulfide (H₂S) were measured using a portable Gas Data Analyzer. The results are set out in Table 2. All gas concentrations are expressed as either percentage (%) or parts per million (ppm).

TABLE 2
Concentration of gaseous components measured
during the biomethane potential test

| | Analysis | |
|---|---|---|
| | Untreated beef cattle | Treated beef cattle |
| | manure | manure |
| Total biogas (L/kg VS) | 398 | 439 |
| Methane (CH₄) % | 69.6 | 70.2 |
| Carbon dioxide (CO₂) % | 28.4 | 27.9 |
| Hydrogen sulfide (H₂S) ppm | 70 | 60 |

In the BMP test the total biogas yield produced using untreated cow manure was 398 L biogas/kg VS, which is in the Low Biogas Yield range. In comparison, total biogas yield from the product of the process of the invention was 439 L biogas/kg VS from manure subjected to the process of the invention, which is in a moderate biogas range. The biogas from the process of the invention included 70.2% methane, indicating an excellent biogas quality. Generally, greater than 60% methane in biogas is considered excellent quality. Hydrogen sulfide (H₂S) was significantly reduced in the treated manure sample, which indicated a better biogas quality from the treated material.

The experimental data for manure from beef cattle demonstrates that when the process of the invention is applied to such manures, higher quantities of biogas and energy can be generated, contributing to lower greenhouse gas emissions.

### Example II

In another example, dairy cow manure was treated with the process of the invention. Untreated and treated samples of the dairy cow manure were obtained. The “untreated” sample corresponds to the animal manure digestate 26, i.e., prior to being subjected to the thermal-alkaline-mechanical process of the invention. The “treated” sample corresponds to the hydrolyzed animal manure digestate 30.

In this example also, the treated sample appears to be more homogeneous than the untreated sample. The results are set out in Tables 3 and 4 below.

TABLE 3
Analysis of untreated and treated dairy cow manure

Analysis

| | Untreated dairy cow | Treated dairy cow |
|---|---|---|
| | manure | manure |
| Total solids (%) | 20.9 | 17.5 |
| Volatile solids (%) | 15.0 | 14.0 |
| Ash (%) | 28.1 | 20.2 |

A BMP test was conducted in the same manner as described above. In the BMP test the total biogas yield produced using untreated dairy cow manure was 186 L biogas/kg VS, whereas total biogas yield from the treated manure was 200 L biogas/kg VS.

The treated sample provided biogas of about 70% methane, indicating an excellent biogas quality (Table 4). Hydrogen sulfide (H₂S) was significantly reduced in the treated dairy cow manure sample, which indicates a better biogas quality from the treated material.

TABLE 4
Concentration of gaseous components measured
during the biochemical methane potential test

Analysis

| | Untreated dairy | Treated dairy |
|---|---|---|
| | cow manure | cow manure |
| Total biogas (L/kg VS) | 186 | 200 |
| Methane (CH₄) % | 68.0 | 70.0 |
| Carbon dioxide (CO₂) % | 31.2 | 30.5 |
| Hydrogen sulfide (H₂S) ppm | 20.0 | 12.5 |

### Example III

Swine manure samples were treated using the process of the invention. Untreated and treated samples of the swine manure were obtained. The “untreated” sample corresponds to the animal manure digestate 26, i.e., prior to being subjected to the thermal-alkaline-mechanical process of the invention. The “treated” sample corresponds to the hydrolyzed animal manure digestate 30.

It was observed that the treated sample was more liquefied and homogeneous than the untreated sample.

Analytical results on the untreated and treated manure samples are shown in Table 5.

TABLE 5
Analysis of untreated and treated swine manure

| Analysis | Untreated swine manure | Treated swine manure |
|---|---|---|
| Total solids (%) | 1.0 | 2.0 |
| Volatile solids (%) | 0.5 | 0.6 |
| Ash (%) | 55.0 | 68.2 |

A BMP test was conducted in the same manner as described above. In the BMP test the total biogas yield produced using untreated swine manure was 387 L biogas/kg VS (Table 6), which is in the Low Biogas Yield range. In comparison, total biogas yield from the treated manure was 693 L biogas/kg VS, which is in a Moderate Biogas Yield range and an improvement by about 80%.

Biogas quality consisted of 66.6% methane, indicating an excellent biogas quality. Hydrogen sulfide (H₂S) was found to be substantially reduced in the treated swine manure sample.

TABLE 6
Concentration of gaseous components measured
during the biochemical methane potential test

| | Analysis | |
|---|---|---|
| | Untreated swine | Treated swine |
| | manure | manure |
| Total biogas (L/kg VS) | 387 | 693 |
| Methane (CH₄) % | 65.1 | 66.6 |
| Carbon dioxide (CO₂) % | 20.8 | 17.6 |
| Hydrogen sulfide (H₂S) ppm | 170 | 67.5 |

### Example IV

Goat manure samples were analyzed and treated with the method of the invention. Untreated and treated samples of the goat manure were obtained. The “untreated” sample corresponds to the animal manure digestate 26, i.e., prior to being subjected to the thermal-alkaline-mechanical process of the invention. The “treated” sample corresponds to the hydrolyzed animal manure digestate 30.

The untreated goat manure sample was quite fibrous and appeared heterogeneous. However, the treated material becomes more homogeneous and liquefied. Table 7 shows analysis of the untreated manure samples.

TABLE 7
Analysis of untreated and treated goat manure

| Analysis | Untreated goat manure | Treated goat manure |
|---|---|---|
| Total solids (%) | 21.7 | 13.8 |
| Volatile solids (%) | 17.0 | 10.7 |
| Ash (%) | 21.7 | 22.7 |

A BMP test was conducted in the same manner as described above. In the BMP test the total biogas yield produced using untreated and treated goat manure was 50 L biogas/KG VS, and 154 L biogas/kg VS, respectively (Table 8.). A substantial improvement in biogas yield after thermal-alkaline-mechanical hydrolysis treatment in the reactor vessel. Biogas from treated sample consisted of 61.0% methane indicating an excellent biogas quality.

TABLE 8
Concentration of gaseous components measured
during the biochemical methane potential test

Analysis

| | Untreated goat | Treated goat |
|---|---|---|
| | manure | manure |
| Total biogas (L/kg VS) | 50 | 154 |
| Methane (CH₄) % | 56.3 | 61.0 |
| Carbon dioxide (CO₂) % | 25.4 | 29.2 |
| Hydrogen sulfide (H₂S) ppm | 20.0 | 25.0 |

From the foregoing, it can be seen that the process of the invention, in particular the thermal-alkaline-mechanical hydrolysis process in the reactor vessel, can result in production of greater quantities of biogas, and biogas of better quality. The effects of the method of the invention appear to vary depending on the species, of the animal, probably at least partly due to different feeds being provided to different species, respectively. Although the digestate of some animal manures include substantial fibrous material, this is addressed by utilizing the mixer device in the reactor vessel, to reduce the fibrous material to relatively small particle sizes. Those skilled in the art would appreciate that the biogas may be converted into a suitable form of energy, ultimately resulting in lower greenhouse gas emissions.

It will be appreciated by those skilled in the art that the invention can take many forms, and that such forms are within the scope of the invention as claimed. The scope of the claims should not be limited by the preferred embodiments set forth in the examples, but should be given the broadest interpretation consistent with the description as a whole.

We claim:

1. A method of processing an animal manure that includes biosolids to provide an output animal manure digestate and an output biogas, the method comprising the steps of:
(a) storing the animal manure for a first preselected time period, at a first predetermined temperature, to at least partially liquefy the biosolids to produce an animal manure digestate, and to generate biogas;
(b) after the first preselected time period, heating the animal manure digestate to a second predetermined temperature for a second preselected time period, causing a pH of the animal manure digestate to be a preselected target pH, and subjecting the animal manure digestate to shearing, to produce a hydrolyzed animal manure digestate; and
(c) after the second preselected time period, storing the hydrolyzed animal manure digestate for a third preselected time period, at a third predetermined temperature, to produce the output animal manure digestate, and to generate the output biogas.

2. The method according to claim 1 in which the first predetermined temperature is between 35° C. and 40° C.

3. The method according to claim 1 in which the first predetermined temperature is between 55° C. and 60° C.

4. The method according to claim 1 in which the first preselected time period is between two and 40 days.

5. The method according to claim 1 in which the second predetermined temperature is between 70° C. and 80° C.

6. The method according to claim 1 in which the preselected target pH is between 8.5 and 9.5.

7. The method according to claim 1 in which the second preselected time period is between five minutes and 15 minutes.

8. The method according to claim 1 in which the third predetermined temperature is between 35° C. and 40° C.

9. The method according to claim 1 in which the third predetermined temperature is between 55° C. and 60° C.

10. The method according to claim 1 in which the third preselected time period is between two and 40 days.

11. A method of processing an animal manure that includes biosolids to provide an output animal manure digestate and an output biogas, the method comprising the steps of:
(a) subjecting the animal manure to a first anaerobic digestion at a first predetermined temperature for a first preselected time period, to at least partially liquefy the biosolids to produce an animal manure digestate, and to generate biogas;
(b) after the first preselected time period, heating the animal manure digestate to a second predetermined temperature for a second preselected time period, causing a pH of the animal manure digestate to be a target pH, and subjecting the animal manure digestate to shearing, to produce a hydrolyzed animal manure digestate; and
(c) after the second preselected time period, subjecting the hydrolyzed animal manure digestate to a second anaerobic digestion at a third predetermined temperature for a third preselected time period, to produce the output animal manure digestate, and to generate the output biogas.

12. A method of processing an animal manure that includes biosolids to provide an output animal manure digestate and an output biogas, the method comprising the steps of:
(a) heating the animal manure to a predetermined hydrolysis temperature for a preselected hydrolysis time period, causing a pH of the animal manure to be a target hydrolysis pH, and subjecting the biosolids to shearing for the preselected hydrolysis time period, to produce a hydrolyzed animal manure digestate; and
(b) after the preselected hydrolysis time period, subjecting the hydrolyzed animal manure digestate to anaerobic digestion at a predetermined anaerobic digestion temperature for a preselected anaerobic digestion time period, to produce the output animal manure digestate, and to generate the output biogas.

13. The method according to claim 12 in which the predetermined hydrolysis temperature is between 70° C. and 80° C.

14. The method according to claim 12 in which the target hydrolysis pH is between 8.5 and 9.5.

15. The method according to claim 12 in which the preselected hydrolysis time period is between five minutes and 15 minutes.

16. The method according to claim 12 in which the predetermined anaerobic digestion temperature is between 35° C. and 40° C.

17. The method according to claim 12 in which the predetermined anaerobic digestion temperature is between 55° C. and 60° C.

18. The method according to claim 12 in which the preselected anaerobic digestion time period is between two and 40 days.
